(19) United States
(12) Patent Application Publication
(10) Pub. No.: US 2012/0046622 A1
(43) Pub. Date: Feb. 23, 2012

(54) METHOD AND APPARATUS FOR MAKING A POROUS BIODEGRADEABLE MEDICAL IMPLANT DEVICE

(76) Inventors: Jianhong ZHAO, Singapore (SG); Xu Li, Singapore (SG); Ge Chen, Singapore (SG); Jia En Low, Singapore (SG); Kui Liu, Singapore (SG)

(21) Appl. No.: 13/187,414

(22) Filed: Jul. 20, 2011

(30) Foreign Application Priority Data
Jul. 20, 2010 (SG) 201005300-7

(51) Int. Cl. A61L 31/16; B29C 45/00; A61M 25/00; C08J 9/26

(52) U.S. Cl. 604/265; 264/49; 425/577; 604/264

(57) ABSTRACT

A method of producing a porous biodegradable medical implant device, the method comprising providing a mixed blend comprising a mixture of at least two biocompatible materials having different degradation or solubility characteristics; molding the mixed blend to produce a molded part; and processing the molded part to remove one of the at least two biocompatible materials by a predetermined amount from the molded part to produce the porous biodegradable medical implant device.

### TECHNICAL FIELD

This application relates generally to medical devices and more particularly to a method and apparatus for forming a porous medical device.

### Background

Porous medical devices can be useful as implants for tissue regeneration and drug delivery, especially in nerve rehabilitation applications. Unfortunately, it is particularly challenging to provide such medical devices with one or more lumens in a manner suitable for mass production. There is therefore a need for methods and apparatus to enable easier mass production of such medical devices, so that the cost of healthcare to patients can be better managed.

### SUMMARY

According to a first exemplary aspect, there is provided a method of producing a porous biodegradable medical implant device, the method comprising providing a mixed blend comprising a mixture of at least two biocompatible materials having different degradation or solubility characteristics; molding the mixed blend to produce a molded part; and processing the molded part to remove one of the at least two biocompatible materials by a predetermined amount from the molded part to produce the porous biodegradable medical implant device.

The providing step may include compounding the at least two biocompatible materials to form the mixed blend.

The compounding may include mixing the at least two biocompatible materials with a medicinal product or additives to form the mixed blend.

One of the at least two biocompatible materials may be a water soluble polymer and another of the at least two biocompatible materials may be a water insoluble polymer.

The blend may contain 10 to 20 wt % of the water soluble polymer.

One the at least two biocompatible materials may be selected from the group consisting of PEG, water soluble triblock copolymer of polyethylene oxide) and poly(propylene oxide), water soluble diblock copolymer of poly(ethylene oxide) and poly(propylene oxide), water soluble poly(propylene oxide), polyvinylpyrrolidone, and polyacrylamide.

Another of the at least two biocompatible materials may be selected from the group consisting of PCL, PCL-PEG block copolymer, PCL-polysiloxane block copolymer, other PCL block copolymers with melting temperatures lower than a threshold temperature, poly(lactic-co-glycolic acid), poly(hydroxybutyrate), and other polyesters and polyester copolymers with a melting temperature lower than a threshold temperature.

The threshold temperature may be about 85° C.

The compounding may be performed at a compounding temperature of between 65° C. and 85° C.

The compounding temperature may be about 80° C.

The molding may include molding the mixed blend at a molding temperature of below 85° C. to produce the molded part.

The molding temperature may be between 65° C. and 85° C.

The molding step may include increasing a length of a mold cavity during injection of the blend into the mold clay.

The processing step may include immersing the molded part in water to remove the predetermined amount of the one of the at least two biocompatible materials to form a leached part. The immersing may be performed at a temperature of around 25° C. to 40° C. for 6 to 12 hours. Alternatively, the immersing may be performed for up to two days.

The method may further comprise a step of freezing the leached part to remove excessive solvent from the leached part to form the porous biodegradable medical implant device.

According to a second exemplary aspect, there is provided an apparatus for forming a molded part for producing a porous biodegradable medical implant device, the apparatus having an interchangeable mold component which includes a first mold component having an elongate hollow body; and a second mold component having an elongate body extending from the first mold component to partially define a cavity for receiving a mixed blend to be molded to produce the molded part, the second mold component being arranged to be retractable within and configured to cooperate with the elongate hollow body of the first mold component.

The first mold component may be further configured to move relative to the second mold component for increasing a length of the mold cavity during injection of the mixed blend into the mold cavity.

The first mold component may be configured to move relative to the second mold component to push the molded part off the second mold component after solidification of the molded part around the second mold component in the mold cavity.

The second mold component may comprise a plurality of longitudinally extending micro grooves.

The second mold component may be configured to form at least one through hole in the molded part along a longitudinal axis of the first mold component.

According to a third exemplary aspect, there is provided a porous biodegradable medical implant device comprising a cylindrical tube having a porous and biodegradable structure and at least one elongate channel disposed therein along a longitudinal axis of the device.

The at least one elongate channel may include an inner surface having a plurality of longitudinally extending micro grooves formed thereon.

The porous biodegradable medical implant device may further comprise at least one drug loaded therein for gradual release of the drug during degradation of the device.

### BRIEF DESCRIPTION OF DRAWINGS

FIG. 1 is a flowchart part illustrating an exemplary embodiment of a method of forming a medical device.

FIG. 2A is a cross-sectional view of an exemplary embodiment of an apparatus for forming a medical device according to the method of FIG. 1;

FIG. 2B is the apparatus of FIG. 2A in an alternative molding configuration;

FIG. 3A is a schematic drawing of an exemplary embodiment of the interchangeable mold components of FIG. 2;

FIGS. 3B and 3C are schematic drawings of the interchangeable mold components of FIG. 3A with one embodiment of a part of FIG. 1.

FIG. 4A is an enlarged schematic drawing of the embodiment of the interchangeable mold components of FIG. 3A;

FIG. 4B is an enlarged schematic drawing of the part of FIGS. 3B and 3C.

FIG. 5A is a perspective view of an exemplary embodiment of a medical device formable using the method of FIG. 1;

FIG. 5B is a microscopic image showing a porous structure of the medical device of FIG. 5A;

FIG. 5C is a cross-sectional view of the medical device of FIG. 5A.

FIG. 6A is a schematic drawing of an exemplary embodiment of the interchangeable mold components of FIG. 2;

FIG. 6B is a schematic drawing of an exemplary embodiment of a part formable using the interchangeable mold components of FIG. 6A.

### DETAILED DESCRIPTION

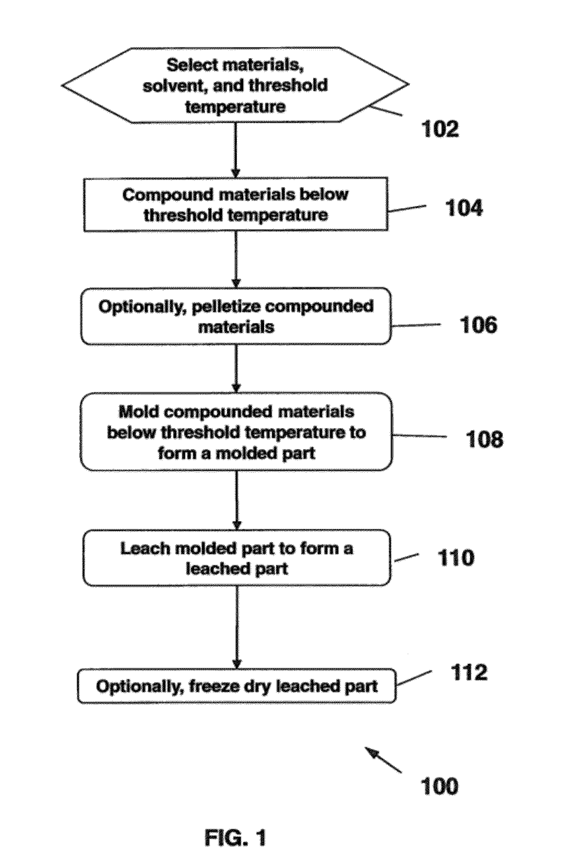

An exemplary embodiment of a method 100 of making a porous biodegradable medical implant device 700 and an exemplary apparatus 200 for making the device 700 will be described with reference to FIGS. 1 to 6B below.

A selecting step 102 of the method 100 includes selecting at least two biocompatible materials characterized by different solubility in a predetermined solvent or -different rates of degradation. Selecting the at least two biocompatible materials may comprise selecting at least one biocompatible first material and at least one biocompatible second material, wherein the at least one biocompatible first material is less soluble in a predetermined solvent than the at least one biocompatible second material, and wherein the at least one first material is also degradable under physiological conditions. Being under physiological conditions refers generally to being implanted or introduced in whole or in part into the body of a living organism such as a human body.

The selecting step 102 further includes selecting a threshold temperature. The threshold temperature is selected to be higher than the melting point of the selected materials, and lower than a temperature above which chemical reactions may occur between the selected materials. The threshold temperature is selected to be lower than a temperature above which at least one of the selected materials will be rendered unsuitable for implantation or introduction in whole or in part into a living organism such as a human being.

The at least one biocompatible first material is selected from a first group consisting of: polyethylene glycol (PEG), water-soluble triblock copolymer of poly(ethylene oxide) and poly(propylene oxide), water-soluble diblock copolymer of poly(ethylene oxide) and poly(propylene oxide), water soluble poly(propylene oxide), polyvinylpyrrolidone, and polyacrylamide.

The at least one biocompatible second material has a different solubility in water from the first biocompatible material, and is selected from a second group consisting of: poly(caprolactone) (PCL), PCL-PEG block copolymer, PCL-polysiloxane block copolymer, poly(lactic-co-glycolic acid), and poly(hydroxybutyrate). The at least one first material and the at least one second material are selected such that they do not chemically react with one another below the selected threshold temperature. The selected at least two biocompatible materials are selected to be chemically inert to one another, that is to say, there will not be chemical reaction between the selected materials under the threshold temperature. It will be understood by one skilled in the molded part that physical interactions, such as any formation of van der Waals bonds between the selected materials, do not constitute chemical reactions.

The method 100 further includes a step 104 of compounding all the materials selected at selecting step 102. The step 104 of compounding involves mixing all the selected at least two biocompatible materials at a compounding temperature that is below the selected threshold temperature and above the melting temperatures of the selected at least two biocompatible materials. Accordingly, the step 104 of compounding, does not involve chemical reactions between the selected materials since it is performed at a temperature below the selected threshold temperature. The step of 104 of compounding is thus a step of physically forming a polymeric mixed blend or mixture of the selected at least two biocompauble materials without chemically reacting any of the materials in the mixed blend. This is necessary in order to retain the distinct solubility characteristics of each of the at least two biocompatible materials, so as to allow one of the at least two biocompatible materials to be subsequently remove in order to form pores in the device 700.

In an exemplary embodiment, water is selected as the predetermined solvent. PEG and PCL are selected as the biocompatible first and second materials respectively. PEG is a biocompatible material that is relatively more soluble in water when compared to PCL. PEG may be chemically or physically cross-linked, and is relatively more soluble in water compared to PCL. PCL is a biocompatible polyester that may be degraded by hydrolysis of its ester linkages under physiological conditions. The PEG has a melting point of 50° C. and the PCL has a melting point of 60° C. Below 85° C., the two materials do not undergo chemical reactions or become unsuitable for incorporation with the human body. Thus, in the exemplary embodiment, the selected threshold temperature is 85° C.

In the exemplary embodiment, the polymeric mixed blend resulting from the compounding step 104 contains 10 to 20 wt % of PEG. The compounding temperature is about 80° C. In other examples, the compounding temperature may range from about 65° C. to about 85° C. It is also envisaged that the compounding temperature may be between 67° C., and 83° C., 70° C. and 80° C. 72° C. and 78° C. etc.

After the compounding step 104, the method 100 further comprises a molding step 108 configured to form a molded part 500 using the mixed blend.

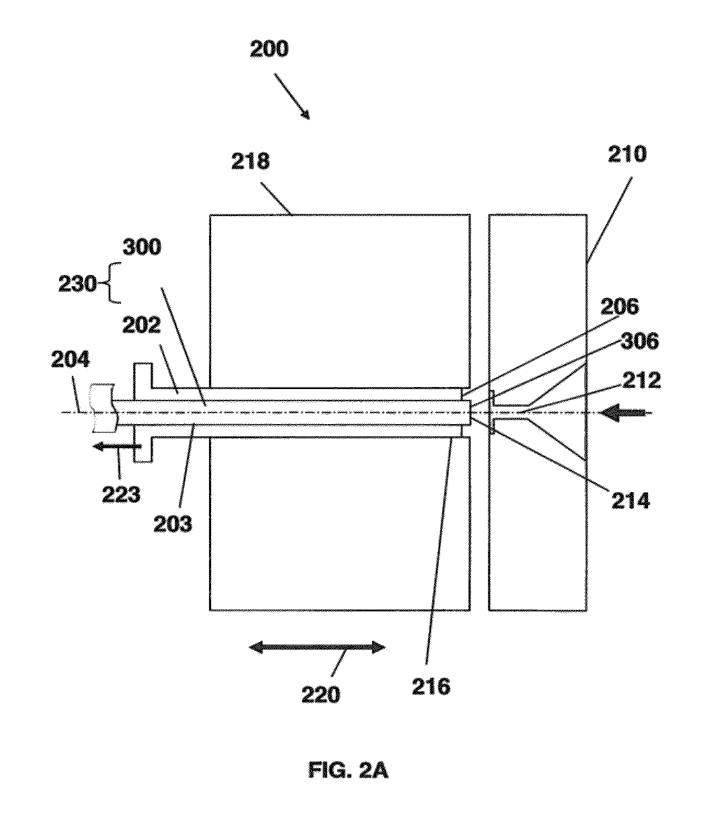

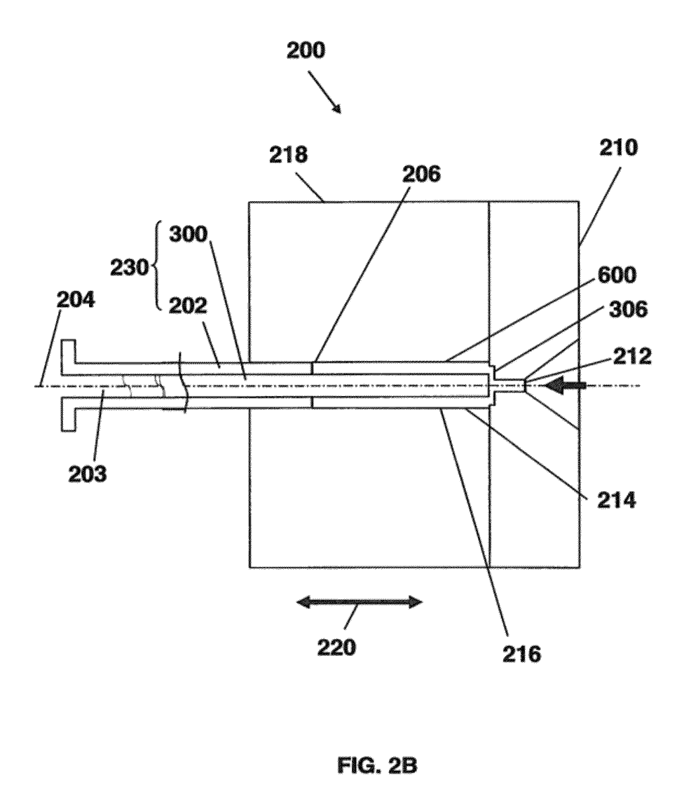

FIG. 2A shows a preferred embodiment of the apparatus 200 configured to carry out the molding step 108 of the method 100. The apparatus 200 is configured for use with an injection molding machine. The apparatus 200 includes a stationary mold component 210 having a conduit or sprue 212 configured to direct and convey a melt 600 to a mold cavity 214. The melt 600 is formed by subjecting a predetermined amount of the mixed blend to a molding temperature that is above the melting point of the mixed blend and below the threshold temperature, thereby ensuring that the distinct solubility characteristics of each of the at least two biocompatible materials are retained after molding 108. In choosing the molding temperature for the molding step 108, it will be understood that the melting point of the mixed blend may vary from case to case, depending on the relative wt % of the selected materials as well as the choice of the selected materials. In some examples, the molding temperature may range from around 65° C. to around 85° C. In the exemplary embodiment, the molding temperature used is 80° C.

The apparatus 200 further comprises a moveable mold component 218 that is configured to contact the stationary mold component 210 by relative movement of the moveable mold component 218 with respect to the stationary mold component 210 in a direction substantially parallel to direction arrow 220. The moveable mold component 218 is provided with the mold cavity 214 into which the melt 600 is introduced through the sprue 212 in the stationary mold component 210 when the stationary mold component 210 and the moveable mold component 218 have been brought into contact with each other. The mold cavity 214 is provided with at least one interchangeable mold component 230, and is preferably cylindrically shaped.

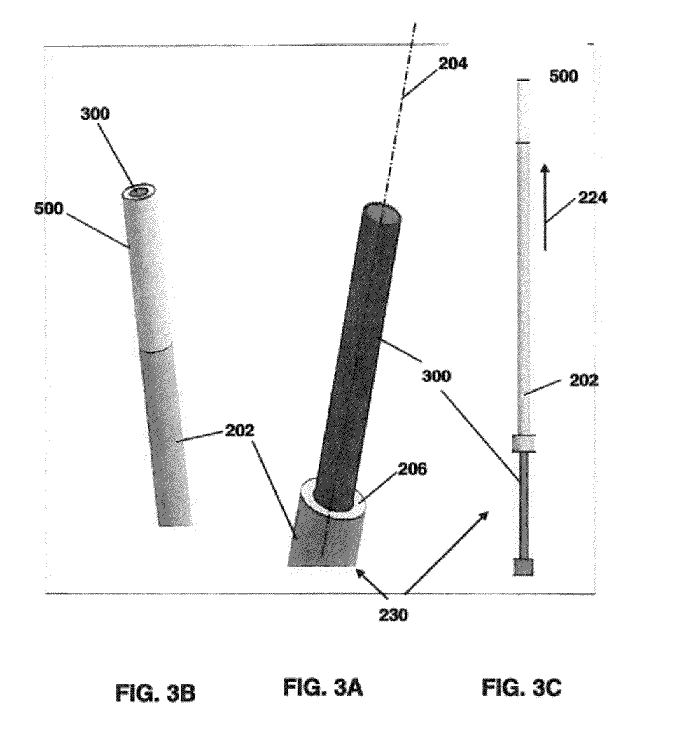

Referring also to FIGS. 3A, 3B and 3C, a preferred embodiment of the at least one interchangeable mold component 230 includes a first mold component 202 and a second mold component 300. The first mold component 202 comprises an elongate hollow body and is configured to slideably engage the mold cavity 214. The first mold component 202 comprises at least one through hole along a longitudinal axis 204 of the first mold component 202 such that the first mold component 202 is in the form of a hollow tube or sleeve and the at least one through hole is a central through hole 203. The second mold component 300 is configured to form at least one elongate channel or hole in the molded part 500 along a longitudinal axis of the molded part 500. The second mold component 300 is configured to slideably engage the longitudinal through hole 203 in the first mold component 202, and is generally rod-shaped.

The first mold component 202 preferably includes an end surface 206 such as an end feature forming section or surface 206 configured to define an end surface of a product or part 500 formable in the mold cavity 214 in the molding step 108. The feature forming surface 206 preferably comprises a surface having a plane perpendicular to the longitudinal axis 204 of the first mold component 202.

In the molding step 108, the second mold component 300 is extending from the first mold component to partially define a cavity for receiving the mixed blend forming the melt 600 to be molded to produce the molded part 500. The second mold component 300 is preferably positioned adjacent the entry of the mold cavity 214 while the end feature forming surface 206 of the first mold component 202 is positioned at a distance from the entry of the mold cavity 214 such that a final mold cavity 214 defining a shape of the part 500 to be formed is defined by the internal surface 216 of the moveable mold component 218, the end feature forming surface 206 and the second mold component 300 as shown in FIG. 2A. During molding 108, the melt 600 is injected into the final mold cavity 214 while both the first mold component 202 and the second mold component 300 are kept stationary as the melt 600 fills the final mold cavity 214 and is allowed to cool to form the part 500. The stationary second mold component 300 thus defines an internal surface of the part 500 being formed while an internal surface 216 of the moveable mold component 218 defines a longitudinal external surface of the part 500. Of the first mold component 202, only the end feature forming surface 206 is in contact with the melt 600 to form the end surface of the part 500.

At the end of the molding step 108, the stationary mold component 210 and the moveable mold component 218 are moved apart and the molded part 500 is ejected. To eject the molded part 500, the first mold component 202 is moved relative to the second mold component 300 in a direction shown by arrow 224 in FIG. 3C, towards the entry of the mold cavity 214 parallel to the direction shown by arrow 220 in FIG. 2A. In this way, the end feature forming surface 206 of the first mold component 202 pushes against the end surface of the molded part 500 to slide the formed part 500 off the second mold component 300 around which the molded part 500 has solidified. The second mold component 300 is thus arranged to be relatively retractable within and configured to cooperate with the elongate hollow body of the first mold component 202 for ejecting the molded part. 500 and for molding the molded part 500 respectively.

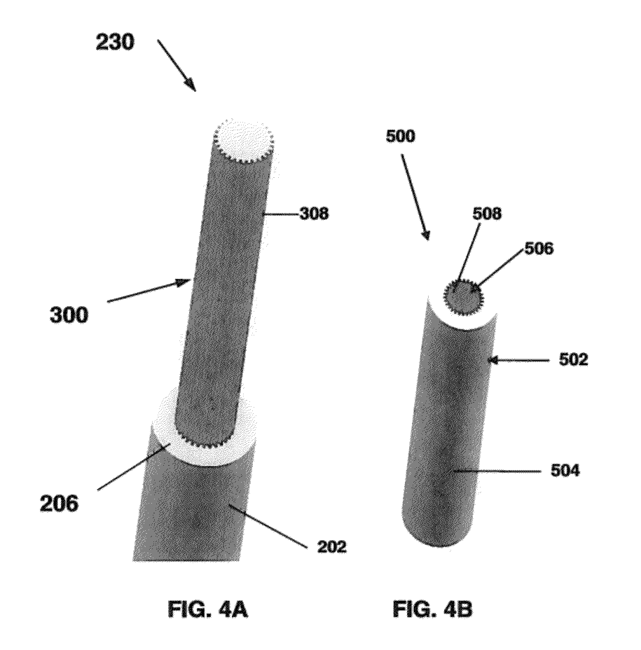

FIG. 4A is an enlarged view of one embodiment of the interchangeable mold component 230 which is used in the molding apparatus 200 of FIG. 2A. The interchangeable mold component 230 includes a first mold component 202 and a generally rod-shaped second mold component 300. The first mold component 202 has an end feature forming surface 206. The second mold component 300 has a surface configuration comprising a surface configuration of micro grooves 308 extending in a generally longitudinal direction on an outer surface of the second mold component 300. The second mold component 300 extends longitudinally from the end feature forming surface 206 of the first mold component 202 during molding 108.

As shown in FIG. 4B, a part 500 producible by the interchangeable mold component 230 of FIG. 4A is a hollow tube 502 having an outer surface 504 defined by the inner wall 216 of the moveable mold component 218 and an inner surface 506 defined by the outer surface of the second mold component 300. The inner surface 506 of the part 500 has a plurality of longitudinally extending micro grooves 508 formed thereon corresponding or complementary to the surface configuration 308 of the second mold component 300.

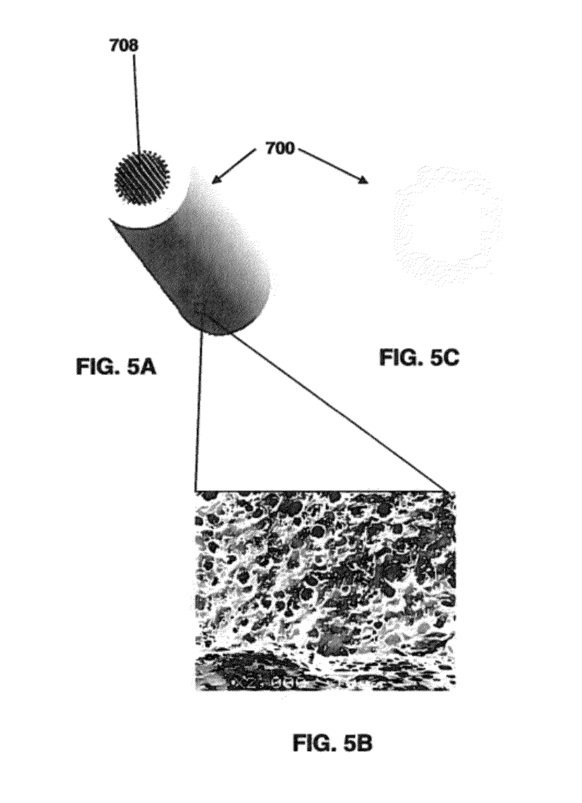

After the molding step 108, the method 100 further comprises processing the molded part 500 to remove one of the at least two biocompatible materials by a predetermined amount from the molded part 500 to produce the porous biodegradable medical implant device 700 having a physical shape and features as shown in FIGS. 5A, 5B and 5C. The device 700 includes micro grooves 708 corresponding to the microgrooves 508 of the molded part 500 formed by the microgrooves 308 on the second mold component 300 in the molding step 108. Performing the processing step 110, 112 after the molding step 108 facilitates mass production of the device 700 with the desired overall dimensions and physical features including the micro grooves. In this manner, the difficulties of creating tubular structures by working with a porous material from the beginning may be circumvented.

The step of processing the molded part 500 includes a leaching step 110. The leaching step 110 involves placing or immersing the molded part 500 in the predetermined solvent under appropriate conditions. The appropriate conditions include a predetermined period of time of immersion of the part 500 until a desired amount of one of the at least two biocompatible materials has been dissolved or leached off by the predetermined solvent. The leaching step 110 is stopped when the device 700 is observed to have a desired surface configuration, for example, having a surface structure as shown in the magnified image of FIG. 5B. Alternatively, the leaching step 110 is stopped when the device 700 has reached a desired degree of porosity. In the exemplary embodiment where the selected biocompatible materials are PEG and PCL and the predetermined solvent is water, the leaching step 110 comprises immersing the molded part 500 comprising PEG and PCL in water for up to two days at a leaching temperature from 25° C. to 40° C. In other examples, the leaching step 110 may be carried out at a leaching temperature of 37° C., for 6 to 12 hours. The leaching temperature is selected to be lower than the threshold temperature.

Optionally, the processing step may further comprise a freeze-drying step 112 after the leaching step 110 to remove residual solvent from the device 700. The freeze-drying step 112 involves subjecting the leached part 500 to carbon dioxide under supercritical conditions to further increase the degree of porosity in the device 700 produced. The freeze-drying step 112 may further be configured to stabilize the porous morphology of the device 700. In some examples pores are formed on the wall of the device 700 from leaching of small molecular weight PEG and evaporation of water during freeze-drying of the molded part 500. Thus, in the method 100 including the freeze-drying step, tubular structures are first molded before porosity is created by sublimation of ice during freeze drying as well as solubilization of trapped water-soluble polymer during immersing in water after the molding step 108.

In the embodiment described above, the device 700 is in the form of a hollow tube with a porous wall having grooves on an internal wall as shown in FIG. 5C. suitable for implantation into a living organism for guiding the growth of nerve cells and other tissues.

Traditionally, the fabrication of porous medical devices is performed in small batches or even by hand. This was previously particularly challenging as porosity tended to introduce irregularities and dimensional inaccuracy in small features. However, variations of the present method and apparatus enable the fabrication of such parts to enjoy the manufacturing efficiencies of high speed manufacturing processes such as injection molding. Additionally, dimensional accuracy, especially in the channels and lumens formed, can be achieved and maintained.

Advantageously, mechanical features such as channels or lumens that may help to prevent the medical devices from collapsing, kinking, or twisting undesirably may be easily formed. Furthermore, with the present method and apparatus with interchangeable parts, it is now easier to produce longitudinally oriented channels in a conduit (such as a medical device with one or more lumens) suitable for facilitating regeneration of nerve cells, or for producing functional conduits for use in kidney dialysis, etc. The channels and other longitudinal features may also increase the surface area available for cell contact. In addition, the low temperatures involved in the method and apparatus described above make it possible to incomorate drugs and other active materials into the medical device without additional post processing after the final parts have been obtained, and for the drugs or active materials to remain active.

To that end, the selecting step 102 of the method 100 may further include selecting at least one active material having a desired active property or nature, such as a medical, therapeutic, or like property, nature or effect. In some embodiments, the active material may be a drug or a bioactive agent. If the selected materials include at least one active material, the threshold temperature is selected to be lower than a temperature at which at least one of the active materials will be affected such that the active material will be rendered less effective in its respective medicinal, therapeutic or active property, nature or effect. Accordingly, where an active material is also selected in the selecting step 102, the compounding step 104 will also include compounding the selected active material (such as a drug or bioactive agent) with the at least two selected biocompatible materials to form a polymeric mixed blend having the selected active material therein.

PCL has a slow degradation rate and better mechanical properties compared with other biopolymers such as polylactide. By introducing porous structures into a medical device comprising PCL, the degradation rate can be changed and even controlled by introducing different degrees of porosities to suit various applications. Porosity facilitates the delivery of drugs, such as growth factors, and of nutrition solution to desired sites. At the same time, desirable mechanical properties can be introduced to prevent conduit collapse. The described exemplary embodiment thus helps to increase the suitability of PCL for use in the fabrication of medical devices

It will be understood that various other combinations of materials and solvent may be selected in accordance with the criteria for selection taught above.

Optionally, the method 100 also includes a pelletizing step 106 after the compounding step 104 and before the molding step 108. In the pelletizing step 106, the polymeric blend (with or without at least one active material) is formed into pellets. This facilitates the provision of predetermined amounts of the compounded materials for the molding step 108, including increasing the ease of feeding a predetermined amount of the compounded materials to a forming machine, such as the stationary mold component of the apparatus 200 as shown in FIGS. 2A and 2B.

In an alternative molding configuration of the molding step 108, for forming a high aspect ratio part 500, the first mold component 202 and the second mold component 300 may be arranged in a configuration in the mold cavity 214 as shown in FIG. 2B before injection of the melt 600. As can be seen in FIG. 2B, the end feature forming surface 206 of the first mold component 202 is positioned adjacent an entry of the mold cavity 214 which is in turn adjacent the sprue 212 in the stationary mold component 210. An end 306 of the second mold component 300 is also positioned adjacent the entry of the mold cavity 214. In this alternative molding configuration, as the melt 600 is injected through the sprue 212 into the mold cavity 214, the melt 600 comes into contact with the end feature forming surface 206 of the first mold component 202. As the melt 600 continues to be injected through the sprue 212, the first mold component 202 is moved back from the entry of the mold cavity 214 relative to the second mold component 300 in the direction shown by arrow 223. The second mold component 300 is kept stationary.

Moving back the first mold component 202 during injection results in an increase in length of the mold cavity 214 around the stationary second mold component 300, thereby allowing a corresponding increase in the volume of melt 600 around the stationary second mold component 300 as the melt 600 is being injected. Movement of the first mold component 202 is stopped when a desired length of the molded part 500 to be formed is reached, as shown in FIG. 2A, where the second mold component 300 extends substantially beyond the end feature forming surface 206 of the first mold component 202.

In the alternative molding configuration, the first mold component 202 is preferably actively moved back at a predetermined rate daring injection of the melt 600. Active movement of the first mold component 202 during injection may optionally be configured to create a flow condition in the mold cavity 214 for facilitating inflow of the melt 600 into the mold cavity 214 smoothly to minimize air trapping.

Thus, by configuring the first mold component 202 to move during injection of the melt 600 in order to facilitate flow of the melt 600 into the mold cavity 214, a high quality high aspect ratio part 500 may be formed having minimal air cavities therein.

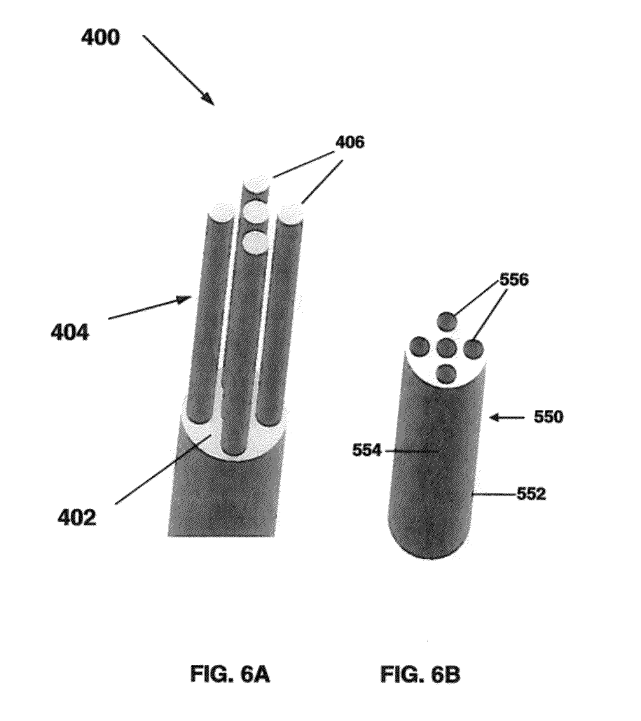

Other shapes, sizes and configurations of molded parts are also envisaged, and may be realized by modifying the interchangeable mold component 230 accordingly. For example, another embodiment of an interchangeable mold component 400 is illustrated in FIG. 6A. The interchangeable mold component 400 includes a generally cylindrical first mold component 401 having an end feature forming surface 402 and a second mold component 404 comprising a plurality of longitudinally extending rod-like elements 406. The inner wall 216 of the moveable mold component 218, the end feature forming surface 402 and the second mold component 404 together define a volume which determines the shape and size of a part 550 formed at the end of the molding step 108.

The second mold component 404 extends longitudinally from the end feature forming surface 402 of the first mold component 401 during molding. The first mold component 401 comprises an equal number of longitudinal through holes configured to slideably engage the plurality of longitudinally extending rod-shaped elements 406 of the second mold component 404 for forming high aspect ratio molded parts 550 or for convenient ejection of the molded part 550 from around the second mold component 404 after molding.

As shown in FIG. 6B, the part 550 may be a cylindrical shape 552 having an outer surface 554 defined by the inner wall 216 of the moveable mold component 218 an end surface 558 defined by the end feature forming surface 402 of the first mold component 401 and a plurality of internal longitudinal through channel or lumen 556 corresponding or complementary to the at least one element 406 of the second mold component 404.

It will also be appreciated that other than micro groovoes 308 described above, the second mold component 300 may have other surface configurations, such as channels and larger grooves, or be smooth or have other predetermined surface finishes.

It is further envisaged that for the apparatus 200, more than one mold cavity 214 may be provided in the moveable mold component 218, and each mold cavity 214 may be provided with at least one interchangeable mold component 230 of any of the variations described above.

Whilst the foregoing description has described exemplary embodiments, it will be understood by those skilled in the technology concerned that many variations in details of design, construction and/or operation may be made without departing from the present invention. For example, the second mold component may comprise any practicable number of rod-shaped elements as may be desired to form a corresponding number of longitudinal through holes in the molded part. The stationary mold component may be moved away from the moveable mold component before and after molding while keeping the moveable mold component stationary. The mold cavity may have a different cross-section other than a uniform circular cross-section to form the cylindrical shape. For example, the mold cavity may have an elliptical cross-section. The cross-section of the mold cavity may also vary in size and shape along the length of the mold cavity according to the shape of the molded part that it is desired to form.

1. A method of producing a porous biodegradable medical implant device, the method comprising:
(i) providing a mixed blend comprising a mixture of at least two biocompatible materials having different degradation or solubility characteristics;
(ii) molding the mixed blend to produce a molded part; and
(iii) processing the molded part to remove one of the at least two biocompatible materials by a predetermined amount from the molded part to produce the porous biodegradable medical implant device.

2. The method of claim 1, wherein the providing step (i) includes compounding the at least two biocompatible materials to form the mixed blend.

3. The method of claim 2, wherein the compounding includes mixing the at least two biocompatible materials with a medicinal product or additives to form the mixed blend.

4. The method of claim 1, wherein one of the at least two biocompatible materials is a water soluble polymer And another of the at least two biocompatible materials is a water insoluble polymer.

5. The method of claim 1, wherein the mixed blend contains 10 to 20 wt % of the water soluble polymer.

6. The method of claim 1, wherein one of the at least two biocompatible materials is selected from the group consisting of PEG, water soluble triblock copolymer of poly(ethylene oxide) and polypropylene oxide) water soluble diblock copolymer of poly(ethylene oxide) and polypropylene oxide), water soluble polypropylene oxide), polyvinylpyrrolidone, and polyacrylamide.

7. The method of claim 6, wherein another of the at least two biocompatible materials is selected from the group consisting of PCL, PCL-PEG block copolymer. PCL-polysiloxane block copolymer, other PCL block copolymers with melting temperatures lower than a threshold temperature, polylactic-co-glycolic acid), poly(hydroxybutyrate), and other polyesters and polyester copolymers with a melting temperature lower than a threshold temperature.

8. The method of claim 7, wherein the threshold temperature is about 85° C.

9. The method of claim 1, wherein the compounding is performed at a compounding temperature of between 65° C. and 85° C.

10. The method of claim 9, wherein the compounding temperature is about 80° C.

11. The method of claim 1, wherein the molding step (ii) includes molding the mixed blend at a molding temperature of below 85° C. to produce the molded part.

12. The method of claim 11, wherein the molding temperature is between 65° C. and 85° C.

13. The method of claim 1, wherein the molding step (ii) includes increasing a length of a mold cavity during injection of the mixed blend into the mold cavity.

14. The method of claim 1, wherein the processing step (iii) includes immersing the molded part in water to remove the predetermined amount of the one of the at least two biocompatible materials to form a leached part.

15. The method of claim 14, wherein the immersing is performed at a temperature of around 23° C. to 40° C. for 6 to 12 hours.

16. The method of claim 14, wherein the immersing is performed for up to two days.

17. The method of claim 14, further comprising a step of freezing the leached part to remove excessive solvent from the leached part to form the porous biodegradable medical implant device.

18. An apparatus for forming a molded part for producing a porous biodegradable medical implant device, the apparatus having: an interchangeable mold component which includes a first mold component having an elongate hollow body; and a second mold component having an elongate body extending from the first mold component to partially define a cavity for receiving a mixed blend to be molded to produce the molded part, the second mold component being arranged to be retractable within and configured to cooperate with the elongate hollow body of the first mold component.

19. The apparatus of claim 18, wherein the first mold component is configured to move relative to the second mold component for increasing a length of the mold cavity during injection of the mixed blend into the mold cavity.

20. The apparatus of claim 18, wherein the first mold component is configured to move relative to the second mold component to push the molded part off the second mold component after solidification of the molded part around the second mold component in the mold cavity.

21. The apparatus of claim 18, wherein the second mold component comprises a plurality of longitudinally extending micro grooves.

22. The apparatus of claim 18, wherein the second mold component is configured to form at least one through hole in the molded part along a longitudinal axis of the first mold component.

23. A porous biodegradable medical implant device comprising a cylindrical tube having a porous and biodegradable structure and at least one elongate channel disposed therein along a longitudinal axis of the device.

24. The porous biodegradable medical implant device of claim 23, wherein the at least one elongate channel includes an inner surface having a plurality of longitudinally extending micro grooves formed thereon.

25. The porous biodegradable medical implant device of claim 23, further comprising at least one drug loaded therein for gradual release of the drug during degradation of the device.
